(19) United States
(12) Patent Application Publication
(10) Pub. No.: US 2022/0346632 A1
(43) Pub. Date: Nov. 3, 2022

(54) IMAGE PROCESSING APPARATUS, IMAGE PROCESSING METHOD, AND NON-TRANSITORY STORAGE MEDIUM STORING COMPUTER PROGRAM

(71) Applicant: OLYMPUS CORPORATION, Tokyo (JP)

(72) Inventors: Hiroshi TANAKA, Tokyo (JP); Takehito HAYAMI, Yokohama-shi (JP); Akihiro KUBOTA, Tokyo (JP); Yamato KANDA, Tokyo (JP); Makoto KITAMURA, Tokyo (JP)

(73) Assignee: OLYMPUS CORPORATION, Tokyo (JP)

(21) Appl. No.: 17/863,869

(22) Filed: Jul. 13, 2022

Related U.S. Application Data
(63) Continuation of application No. PCT/JP2020/001868, filed on Jan. 21, 2020.

(51) Int. Cl. A61B 1/00

(52) U.S. Cl. CPC A61B 1/000095; A61B 1/0005; A61B 1/0002

(57) ABSTRACT

An image processing apparatus includes an input unit, an estimation unit, an acquisition unit, a production unit, and a display control unit. The input unit acquires examination information including an endoscope image. The estimation unit estimates an image pickup site based on the endoscope image. The acquisition unit acquires, from the examination information, image pickup information corresponding to the endoscope image and indicating a state of at least one of an endoscope or a subject when the endoscope image is picked up. The production unit produces a model map and associates the image pickup information with a virtual site based on a result of the estimation by the estimation unit. The display control unit controls a display device.

### CROSS REFERENCE TO RELATED APPLICATION

This application is a continuation application of PCT/JP2020/001868 filed on Jan. 21, 2020, the entire contents of which are incorporated herein by this reference.

### BACKGROUND OF THE INVENTION

### 1. Field of the Invention

The present invention relates to an image processing apparatus, an image processing method, and a non-transitory storage medium storing a computer program that are capable of causing a display device to display information or a condition related to an endoscope image.

### 2. Description of the Related Art

Recently, an endoscope apparatus has been widely used in medical and industrial fields. An endoscope apparatus used in medical fields includes an elongated insertion portion that is inserted into a human body, and the endoscope apparatus is widely used in organ observation, medical treatment using a treatment instrument, surgical operation under endoscope observation, and the like.

In medical fields, medical inspection using an endoscope apparatus is performed on the same organ a plurality of times in some cases. Consider a case in which medical inspection using an endoscope apparatus is performed twice. Hereinafter, the first medical inspection is referred to as a primary medical inspection, and the second medical inspection is referred to as a secondary medical inspection. In the secondary medical inspection, for example, image pickup is performed at a site at which image pickup could not be performed in the primary medical inspection. In order to prevent image pickup omission in the secondary medical inspection, it is desirable to identify a site at which image pickup is performed in the primary medical inspection and a site at which image pickup is not performed.

Japanese Patent Application Laid-Open Publication No. 2018-50890 discloses an image display apparatus configured to generate, from an image photographed by an endoscope apparatus, a map image indicating photographed and unphotographed regions of a photographing target organ. With this image display apparatus, it is possible to identify a site at which image pickup is performed in the primary medical inspection and a site at which image pickup is not performed.

### SUMMARY OF THE INVENTION

An image processing apparatus according to an aspect of the present invention includes a processor, in which the processor is configured to: acquire an endoscope image obtained through image pickup of a subject by an endoscope; estimate an image pickup site in the subject of the endoscope image; associate the image pickup site of the endoscope image with a site corresponding to the image pickup site on an organ model map based on a result of the estimation, the model map including an image pickup condition determined for each site on the map; output the model map and the endoscope image to a monitor; and output, to the monitor, the image pickup condition associated with the site on the model map, the site corresponding to the image pickup site of the endoscope image outputted on the monitor.

An image processing method according to an aspect of the present invention is a method in which a processor is configured to: acquire an endoscope image obtained through image pickup of a subject by an endoscope; estimate an image pickup site in the subject of the endoscope image; associate, based on a result of the estimation, the image pickup site with an organ model map including an image pickup condition defined in advance which is associated with a particular site; output the model map and the endoscope image to a monitor; and output the image pickup condition in the model map to the monitor, the image pickup condition being associated with an image pickup position of the endoscope image outputted on the monitor.

A non-transitory storage medium storing a computer program according to an aspect of the present invention causes a computer to: acquire an endoscope image obtained through image pickup of a subject by an endoscope; estimate an image pickup site in the subject of the endoscope image; associate, based on a result of the estimation, the image pickup site with an organ model map including an image pickup condition defined in advance which is associated with a particular site; and output the model map and the endoscope image to a monitor and output the image pickup condition in the model map to the monitor, the image pickup condition being associated with an image pickup position of the endoscope image outputted on the monitor.

### BRIEF DESCRIPTION OF THE DRAWINGS

FIG. 1 is a functional block diagram illustrating a configuration of an image processing apparatus according to one embodiment of the present invention;

FIG. 2 is a functional block diagram illustrating an example of a hardware configuration of the image processing apparatus according to one embodiment of the present invention;

FIG. 3 is an explanatory diagram illustrating a first use example of the image processing apparatus according to one embodiment of the present invention;

FIG. 4 is an explanatory diagram illustrating a second use example of the image processing apparatus according to one embodiment of the present invention;

FIG. 5 is a flowchart illustrating an image processing method according to one embodiment of the present invention;

FIG. 6 is an explanatory diagram illustrating a first example of display contents according to one embodiment of the present invention;

FIG. 7 is an explanatory diagram illustrating a second example of display contents according to one embodiment of the present invention;

FIG. 8 is an explanatory diagram illustrating a third example of display contents according to one embodiment of the present invention; and

FIG. 9 is an explanatory diagram illustrating a fourth example of display contents according to one embodiment of the present invention.

### DETAILED DESCRIPTION OF THE PREFERRED EMBODIMENT

An embodiment of the present invention will be described below with reference to the accompanying drawings.

### Configuration of Image Processing Apparatus

First, a configuration of an image processing apparatus according to one embodiment of the present invention will be described below. An image processing apparatus 1 according to the present embodiment is an image processing apparatus configured to process an endoscope image generated through image pickup of a subject by an endoscope used in medical fields. In the present embodiment, in particular, the subject is an organ such as stomach or large intestine. The endoscope image is a color image including a plurality of pixels and having pixel values corresponding to a red (R) wavelength component, a green (G) wavelength component, and a blue (B) wavelength component, respectively, at each of the plurality of pixels.

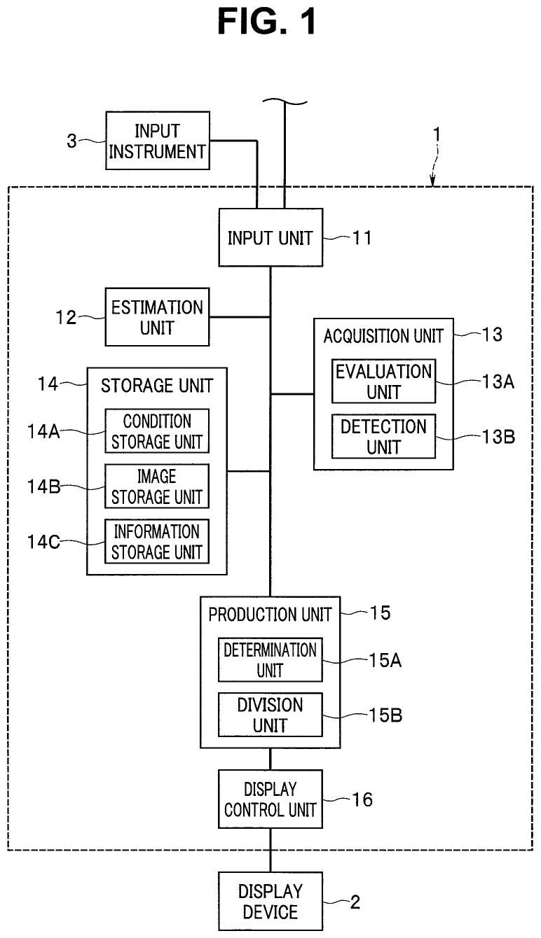

FIG. 1 is a functional block diagram illustrating a configuration of the image processing apparatus 1 according to the present embodiment. The image processing apparatus 1 includes an input unit 11, an estimation unit 12, an acquisition unit 13, a storage unit 14, a production unit 15, and a display control unit 16. The input unit 11 acquires examination information including the endoscope image.

The estimation unit 12 estimates, based on the endoscope image, an image pickup site as a site of the subject, in other words, the organ subjected to image pickup by the endoscope. The estimation of the image pickup site by the estimation unit 12 is performed as image analysis of the endoscope image. For example, when the subject is a stomach, the estimation unit 12 performs image analysis of the endoscope image to estimate whether the image pickup site is, for example, a cardia, a gastric fundus, a gastric body, a lesser curvature, a greater curvature, a gastric antrum, a gastric corner, or a pylorus. When the subject is a large intestine, the estimation unit 12 performs image analysis of the endoscope image to estimate whether the image pickup site is, for example, a rectum, a sigmoid colon, a descending colon, a transverse colon, an ascending colon, or a cecum.

Image analysis may use, for example, pattern matching or machine learning. For example, when the subject is stomach or large intestine, machine learning may be performed by using endoscope images classified for each above-described site. Machine learning may be performed by the estimation unit 12 or a non-illustrated machine learning unit configured to execute machine learning. The estimation unit 12 estimates the image pickup site by using a learning result of machine learning.

Similarly to image analysis at the estimation unit 12, image analysis at a constituent component other than the estimation unit 12 to be described later uses, for example, pattern matching or machine learning.

The acquisition unit 13 acquires, from the examination information, image pickup information corresponding to the endoscope image and indicating a state of at least one of the endoscope or the subject, in other words, the organ when the endoscope image is picked up. The examination information includes, in addition to the endoscope image, system information related to operation of the endoscope. The acquisition of the image pickup information by the acquisition unit 13 is performed as at least one of acquisition of the image pickup information from the system information or acquisition of the image pickup information by performing image analysis of the endoscope image.

The examination information further includes time-point information on a time point when the endoscope image is picked up. The acquisition unit 13 acquires the time-point information as the image pickup information from the examination information.

The acquisition unit 13 includes an evaluation unit 13A configured to evaluate image quality of the endoscope image by performing image analysis of the endoscope image, and a detection unit 13B configured to detect anomaly at the image pickup site by performing image analysis of the endoscope image. Operation of the acquisition unit 13, the evaluation unit 13A, and the detection unit 13B will be described later in more detail.

The storage unit 14 includes a condition storage unit 14A, an image storage unit 14B, and an information storage unit 14C. The condition storage unit 14A stores, for virtual sites to be described later, initial image pickup conditions defined in advance, the initial image pickup conditions being conditions for image pickup of the subject, in other words, the organ. The initial image pickup conditions may be determined by performing image analysis of the endoscope image or may be set by a user. When the initial image pickup conditions are determined by performing image analysis of the endoscope image, the initial image pickup conditions may be determined by the same method as a method by which image pickup conditions are determined by a determination unit to be described later.

In an example illustrated in FIG. 1, an input instrument 3 that is operated by the user is connected to the image processing apparatus 1. The input instrument 3 is constituted by a keyboard, a mouse, a touch panel, or the like. The input unit 11 acquires an operation content inputted to the input instrument 3. When the initial image pickup conditions are set by the user, the user can set the initial image pickup conditions by operating the input instrument 3.

The image storage unit 14B stores the endoscope image acquired by the input unit 11. Note that when the image quality of the endoscope image is evaluated by the evaluation unit 13A, the image storage unit 14B associates and stores a result of the evaluation by the evaluation unit 13A and the endoscope image. The information storage unit 14C stores the image pickup information acquired by the acquisition unit 13.

The production unit 15 produces a model map into which the subject, in other words, the organ is virtualized, and associates the image pickup information with a virtual site corresponding to the image pickup site on the model map based on a result of the estimation by the estimation unit 12, in other words, a result of the estimation of the image pickup site. Specifically, the production unit 15 associates, with a virtual site corresponding to the image pickup site estimated by performing image analysis of any endoscope image, the image pickup information acquired from the examination information including the endoscope image.

The model map may be a schema diagram of the organ or a 3D model diagram of the organ. The number of virtual sites is plural.

The production unit 15 includes a determination unit 15A and a division unit 15B. The determination unit 15A determines, for the virtual sites, image pickup conditions for image pickup of the subject, in other words, the organ. The determination unit 15A may determine the image pickup conditions by performing image analysis of the endoscope image or may determine the image pickup conditions by comparing, for each virtual site, the image pickup information and the initial image pickup conditions.

The division unit 15B divides a virtual site into a plurality of subsites as necessary. For example, when mutually different conditions are determined for a plurality of respective regions included in one virtual site by the determination unit 15A, the division unit 15B divides the one virtual site into a plurality of subsites. The plurality of subsites may be the same as or different from the plurality of regions. The determination unit 15A determines the above-described mutually different conditions as image pickup conditions for the plurality of respective subsites.

Operation of the determination unit 15A and the division unit 15B will be described later in more detail.

In the example illustrated in FIG. 1, a display device 2 for displaying the model map produced by the production unit 15 is connected to the image processing apparatus 1. The display device 2 includes a display unit constituted by a liquid crystal panel.

The display control unit 16 causes the display device 2 to display the endoscope image acquired by the input unit 11, the image pickup information acquired by the acquisition unit 13, the image pickup conditions determined by the determination unit 15A, and the like. In the present embodiment, in particular, the display control unit 16 may control the display device 2 as described below.

When the determination unit 15A determines the image pickup conditions by comparing the image pickup information and the initial image pickup conditions, the display control unit 16 may cause the display device 2 to display at least one of a result of the comparison by the determination unit 15A or at least some of the image pickup conditions determined by the determination unit 15A.

The display control unit 16 may read, from the image storage unit 14B, a plurality of endoscope images that correspond to one image pickup site and at least part of the image pickup information corresponding to which is mutually different, and may read the plurality of pieces of image pickup information corresponding to the plurality of endoscope images from the information storage unit 14C. Then, the display control unit 16 may cause the display device 2 to display at least one of the plurality of endoscope images or the plurality of pieces of image pickup information.

The display control unit 16 may read, from the image storage unit 14B, an endoscope image, the image quality of which is evaluated to be high by the evaluation unit 13A from among a plurality of endoscope images corresponding to one image pickup site, and may cause the display device 2 to display the read endoscope image.

When anomaly at an image pickup site is detected by the detection unit 13B, the display control unit 16 may cause the display device 2 to display a result of the detection by the detection unit 13B so that details of the anomaly can be checked.

The display control unit 16 may cause the display device 2 to display, on the model map based on the time-point information acquired by the acquisition unit 13, an image pickup route when a plurality of endoscope images are picked up.

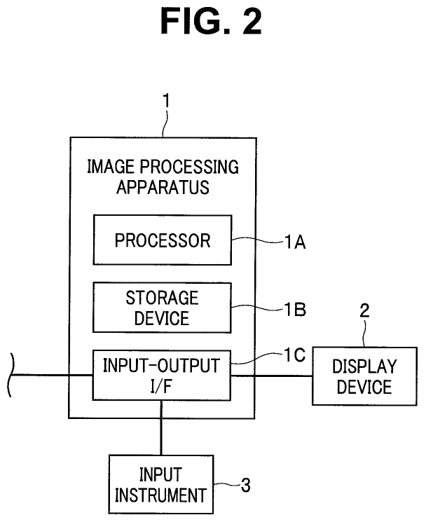

A hardware configuration of the image processing apparatus 1 will be described below with reference to FIG. 2. FIG. 2 is an explanatory diagram illustrating an example of the hardware configuration of the image processing apparatus 1. In the example illustrated in FIG. 2, the image processing apparatus 1 is configured as a computer including a processor 1A, a storage device 1B, and an input-output interface (hereinafter referred to as an input-output I/F) 1C. The processor 1A is constituted by, for example, a central processing unit (hereinafter referred to as a CPU). The storage device 1B is constituted by a storage device such as a RAM, a ROM, a flash memory, or a hard disk device. The input-output I/F 1C is used to perform signal transmission-reception between the image processing apparatus 1 and outside.

The processor 1A is used to execute functions of the input unit 11, the estimation unit 12, the acquisition unit 13, the production unit 15, the display control unit 16, and the like as constituent components of the image processing apparatus 1. The storage device 1B stores an image processing program as a software program for these functions. Each function is implemented as the processor 1A reads and executes the image processing program from the storage device 1B. The storage device 1B stores a plurality of software programs including the above-described image processing program.

Functions of the storage unit 14 as a constituent component of the image processing apparatus 1, in other words, functions of the condition storage unit 14A, the image storage unit 14B, and the information storage unit 14C are basically implemented by a non-transitory rewritable storage device such as a flash memory or a hard disk device in the storage device 1B. The above-described non-transitory rewritable storage device stores initial image pickup conditions, endoscope images, and image pickup information.

Note that the hardware configuration of the image processing apparatus 1 is not limited to the above-described example. For example, the processor 1A may be configured as a field programmable gate array (FPGA). In this case, at least some of a plurality of constituent components of the image processing apparatus 1 are configured as circuit blocks in the FPGA. Alternatively, the plurality of constituent components of the image processing apparatus 1 may be configured as individual electronic circuits.

At least part of the image processing program may be stored in a non-illustrated external storage device or storage medium. In this case, at least some of functions of the image processing apparatus 1 are implemented as the processor 1A reads and executes at least part of the image processing program from the external storage device or storage medium. The external storage device may be, for example, a storage device of another computer connected to a computer network such as a LAN or the Internet. The storage medium may be an optical disk such as a CD, a DVD, or a Blu-ray Disc or may be a flash memory such as a USB memory.

Some of the plurality of constituent components of the image processing apparatus 1 may be implemented by what is called cloud computing. In this case, part of the functions of the image processing apparatus 1 is implemented as another computer connected to the Internet executes part of the image processing program and the image processing apparatus 1 acquires a result of the execution. A hardware configuration of the above-described other computer is the same as the hardware configuration of the image processing apparatus 1 illustrated in FIG. 2. It can be described that the above-described other computer constitutes part of the image processing apparatus 1.

### Use Examples of Image Processing Apparatus

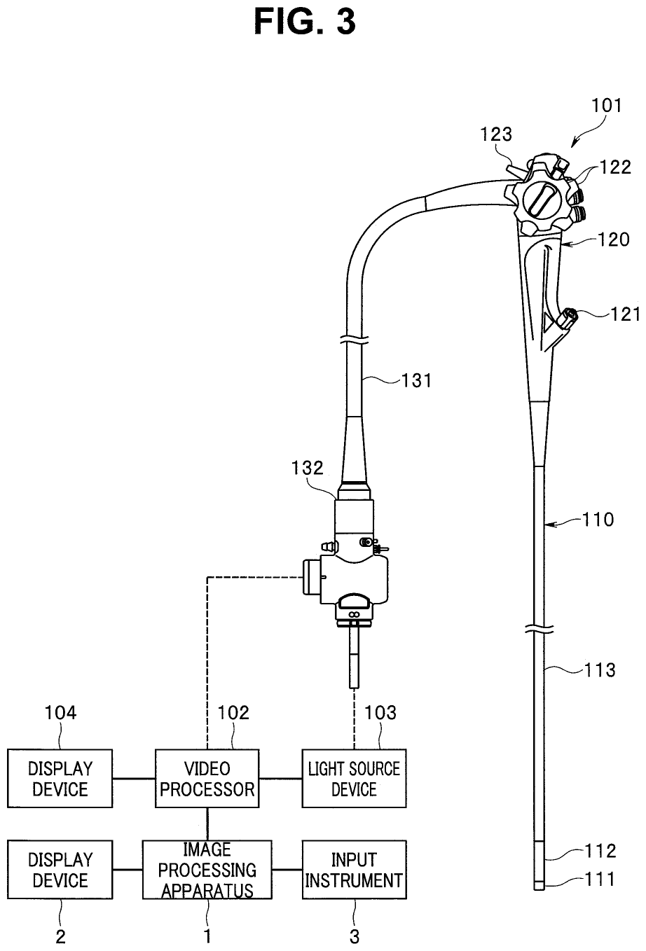

First and second use examples of the image processing apparatus 1 will be described below. The first use example will be described first. FIG. 3 is an explanatory diagram illustrating the first use example of the image processing apparatus 1. FIG. 3 illustrates an endoscope 101, a video processor 102, a light source device 103, and a display device 104 in addition to the image processing apparatus 1, the display device 2, and the input instrument 3 illustrated in FIG. 1. The image processing apparatus 1, the light source device 103, and the display device 104 are connected to the video processor 102.

The endoscope 101 includes an insertion portion 110 inserted into a subject, an operation portion 120 continuously provided at a proximal end of the insertion portion 110, a universal cord 131 extending from the operation portion 120, and a connector 132 provided at a distal end of the universal cord 131. The connector 132 is connected to the video processor 102 and the light source device 103.

The insertion portion 110 includes a distal end portion 111 having an elongated shape and positioned at a distal end of the insertion portion 110, a bending portion 112 that is freely bendable, and a flexible tube portion 113. The distal end portion 111, the bending portion 112, and the flexible tube portion 113 are coupled to each other in the stated order from the distal end side of the insertion portion 110.

A non-illustrated image pickup apparatus is provided at the distal end portion 111. The image pickup apparatus is electrically connected to the video processor 102 through a non-illustrated cable provided in the endoscope 101 and a non-illustrated cable connecting the connector 132 and the video processor 102. The image pickup apparatus includes an observation window positioned at a distalmost end, an image pickup device constituted by a CCD, a CMOS, or the like, and a plurality of lenses provided between the observation window and the image pickup device. At least one of the plurality of lenses is used to adjust optical magnification.

The image pickup device generates an image pickup signal obtained through photoelectric conversion of an optical image of a subject, in other words, organ imaged on an image pickup plane, and outputs the generated image pickup signal to the video processor 102. The video processor 102 generates an image signal by providing predetermined image processing to the image pickup signal and outputs the generated image signal to the display device 104. The display device 104 includes a display unit constituted by a liquid crystal panel. The display device 104 is a device for displaying, as an endoscope image, an image picked up by the image pickup apparatus, in other words, an image pickup signal, and displays, as an endoscope image, the image signal generated by the video processor 102.

In addition, a non-illustrated illumination window is provided at the distal end portion 111. The light source device 103 is controlled by the video processor 102 and generates illumination light. The illumination light generated by the light source device 103 is transmitted to the illumination window through a non-illustrated light guide cable connecting the light source device 103 and the connector 132 and through a non-illustrated light guide provided in the endoscope 101, and is emitted to the subject, in other words, the organ through the illumination window. The light source device 103 can generate, as the illumination light, for example, white light (hereinafter referred to as WLI) that is normal light, and narrow-band light (hereinafter referred to as NBI) that is special light.

The distal end portion 111 may be further provided with a first sensor configured to measure a distance between the distal end portion 111 and an object, and a second sensor configured to detect a tilt angle of the distal end portion 111.

The operation portion 120 is provided with, for example, a treatment-instrument insertion port 121 communicating with a non-illustrated treatment-instrument insertion channel provided in the insertion portion 110, a plurality of bending operation knobs 122 for bending the bending portion 112 of the insertion portion 110, and a zoom lever 123 for moving the lenses of the image pickup apparatus to adjust the optical magnification. A treatment-instrument guide-out port that is an opening portion of the treatment-instrument insertion channel is provided at the distal end portion 111 of the insertion portion 110. A treatment instrument such as a forceps or a puncture needle is introduced into the treatment-instrument insertion channel through the treatment-instrument insertion port 121 and guided out of the treatment-instrument guide-out port.

In an example illustrated in FIG. 3, the video processor 102 outputs examination information. The examination information outputted from the video processor 102 includes an endoscope image, system information, and time-point information. The video processor 102 outputs, as the endoscope image, the image signal generated by the video processor 102. The video processor 102 may output, as the endoscope image (image signal), an image enlarged through the lenses of the image pickup apparatus and an electronically enlarged image obtained by cutting out a central part of the endoscope image and then performing interpolation or the like. The system information outputted from the video processor 102 includes information on magnification (hereinafter referred to as optical magnification) through the lenses and information on magnification (hereinafter referred to as electronic magnification) of the electronically enlarged image.

The system information outputted from the video processor 102 further includes information on kind and light quantity of the illumination light generated by the light source device 103. When the above-described first and second sensors are provided at the distal end portion 111, the system information outputted from the video processor 102 further includes information on detected values by the first and second sensors.

The input unit 11 (refer to FIG. 1) of the image processing apparatus 1 acquires, at a predetermined timing, the examination information outputted from the video processor 102. The predetermined timing may be a timing at which an endoscope image is generated or may be a timing at which the input instrument 3 is operated by the user to acquire the examination information. In the latter case, the examination information is held by the video processor 102 or a non-illustrated storage device until a timing at which the input unit 11 starts acquisition of the examination information.

Note that, in the example illustrated in FIG. 3, the image processing apparatus 1 may use, in place of the display device 2, the display device 104 connected to the video processor 102. Specifically, the display control unit 16 (refer to FIG. 1) of the image processing apparatus 1 may cause the display device 104 to display an endoscope image acquired by the input unit 11, image pickup information acquired by the acquisition unit 13, image pickup conditions determined by the determination unit 15A, and the like. In this case, an endoscope image picked up by the endoscope 101 and display contents determined by the display control unit 16 are displayed on the display device 104. The display device 104 may simultaneously display the endoscope image and the display contents or may switch display of the endoscope image and display of the display contents.

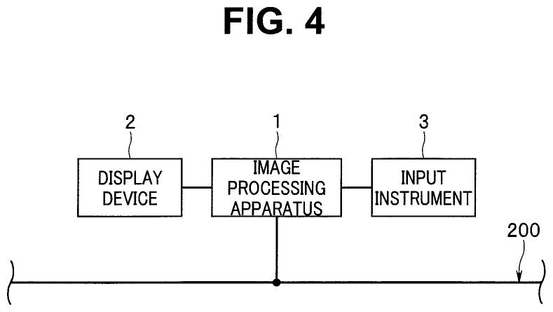

A second example will be described below. FIG. 4 is an explanatory diagram illustrating a second use example of the image processing apparatus 1. In the second use example, the image processing apparatus 1 is connected to a computer network 200 such as a LAN or the Internet. The image processing apparatus 1 may be installed in a medical examination room or an operation room in which, for example, the endoscope 101 and the video processor 102 (refer to FIG. 3) are installed, or may be installed in a room other than the medical examination room and the operation room.

The input unit 11 (refer to FIG. 1) of the image processing apparatus 1 acquires examination information held by the video processor 102 or the non-illustrated storage device through the computer network 200. The non-illustrated storage device may be a storage device of another computer connected to the computer network 200.

### Operation of Acquisition Unit

The operation of the acquisition unit 13 of the image processing apparatus 1 will be described below in detail. As described above, acquisition of image pickup information by the acquisition unit 13 is performed as at least one of acquisition of image pickup information from system information or acquisition of image pickup information by performing image analysis of an endoscope image. Specifically, the acquisition unit 13 acquires, as image pickup information, at least one piece of information from among a plurality of pieces of information such as information on the optical magnification, information on the electronic magnification, information on the kind and light quantity of the illumination light generated by the light source device 103, information on the time point, and information on the detected values by the first and second sensors, which are included in the system information.

An endoscope image includes not only the object but also pigment sprayed as markers and a treatment instrument in some cases. Furthermore, an aspect of the object included in the endoscope image can vary in accordance with the distance between the distal end portion 111 (refer to FIG. 3) and the object and an angle between the distal end portion 111 and the object. Thus, it can be described that the endoscope image includes, in addition to an image of the object, a plurality of pieces of information such as information on whether a region is sprayed with pigment, information on existence and kind of a treatment instrument, information on the distance between the distal end portion 111 and the object (hereinafter referred to as distance information), and information on the angle between the distal end portion 111 and the object (hereinafter referred to as angle information). The acquisition unit 13 acquires, as image pickup information, at least one piece of information from among the above-described plurality of pieces of information by performing image analysis of the endoscope image. Note that, when the above-described first sensor is provided at the distal end portion 111, the distance information may be acquired by using information on the detected value of the first sensor in addition to a result of the image analysis. Similarly, when the above-described second sensor is provided at the distal end portion 111, the angle information may be acquired by using information on the detected value of the second sensor in addition to the result of the image analysis.

As described above, the acquisition unit 13 includes the evaluation unit 13A configured to evaluate the image quality of an endoscope image by performing image analysis of the endoscope image. For example, an image with less blurring, bokeh, saturation, and the like can be thought to have high visibility and high image quality. Thus, the image quality of the endoscope image can be evaluated by quantitatively evaluating the image visibility. Typically, when intensity of an edge of an image is expressed in a magnification relative to a threshold value (hereinafter referred to as a threshold magnification) as a luminance change limit for visual recognizability, the image visibility is higher as the threshold magnification is higher. Thus, the image visibility can be quantitatively evaluated by calculating the threshold magnification for each endoscope image. The image visibility, in other words, viewing easiness is expressed by using, for example, a linear expression having logarithm of the threshold magnification as a variable. The result of the evaluation by the evaluation unit 13A is stored as image pickup information in the information storage unit 14C of the storage unit 14. The image storage unit 14B of the storage unit 14 associates and stores the result of the evaluation by the evaluation unit 13A, which is stored in the information storage unit 14C, and the endoscope image.

As described above, the acquisition unit 13 includes the detection unit 13B configured to detect anomaly at an image pickup site by performing image analysis of an endoscope image. The detection unit 13B detects, for example, a lesion or bleeding as the anomaly at the image pickup site. In particular, a well-known lesion detection algorithm specialized for lesion detection may be used for the lesion detection. The anomaly at the image pickup site is stored as the image pickup information in the information storage unit 14C of the storage unit 14.

### Operation of Determination Unit and Division Unit

The operation of the determination unit 15A and the division unit 15B of the production unit 15 of the image processing apparatus 1 will be described below in detail. As described above, the determination unit 15A determines image pickup conditions for virtual sites. The image pickup conditions may change depending on a plurality of factors such as difference between sites of the organ, existence of anomaly such as a lesion, the distance between the distal end portion 111 and the object, and the angle between the distal end portion 111 and the object. When the image pickup conditions are determined by performing image analysis of an endoscope image, for example, machine learning may be performed on a relation between a plurality of factors and elements of the endoscope image that change due to the plurality of factors. The machine learning may be performed by the determination unit 15A or may be performed by a non-illustrated machine learning unit configured to execute the machine learning. The determination unit 15A determines the image pickup conditions by using a learning result of the machine learning.

When the image pickup conditions are determined by comparing image pickup information and initial image pickup conditions for each virtual site and the image pickup information does not satisfy the initial image pickup conditions, the determination unit 15A determines image pickup conditions with which the initial image pickup conditions are satisfied. The determination unit 15A may additionally determine, for a virtual site corresponding to an image pickup site at which anomaly such as a lesion is detected, image pickup conditions for detailed observation of anomaly such as a lesion irrespective of whether the image pickup information satisfies the initial image pickup conditions. Specifically, for example, the determination unit 15A may additionally determine, as an image pickup condition, use of NBI as the illumination light or increase of the optical magnification or the electronic magnification.

As described above, the division unit 15B divides a virtual site into a plurality of subsites as necessary. The division unit 15B divides, into a plurality of subsites, for example, a virtual site corresponding to an image pickup site at which anomaly such as a lesion is detected as described above. In this case, the division unit 15B may perform division into a subsite including anomaly and a subsite not including anomaly. When a virtual site is divided in this manner, the determination unit 15A may additionally determine, for a subsite including anomaly, for example, image pickup conditions for detailed observation of anomaly such as a lesion. The determination unit 15A may determine, for a subsite not including anomaly, for example, the same image pickup conditions as initial image pickup conditions.

### Image Processing Method

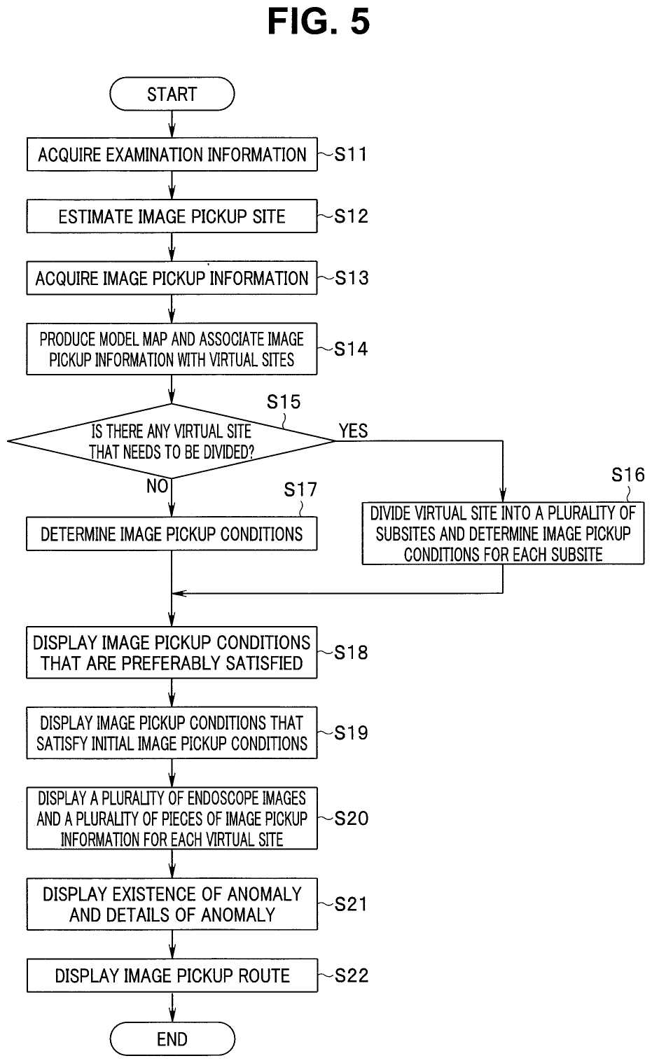

An image processing method according to the present embodiment will be described below with reference to FIG. 5. FIG. 5 is a flowchart illustrating the image processing method according to the present embodiment. In the image processing method according to the present embodiment, first, the input unit 11 acquires examination information (step S11). Subsequently, the estimation unit 12 estimates an image pickup site based on an endoscope image (step S12). Subsequently, the acquisition unit 13 acquires image pickup information from the examination information (step S13). Subsequently, the production unit 15 produces a model map and associates the image pickup information with a virtual site based on a result of the estimation by the estimation unit 12 (step S14).

Subsequently, the determination unit 15A of the production unit 15 provisionally determines image pickup conditions for virtual sites and determines, based on the provisionally determined image pickup conditions, whether there is any virtual site that needs to be divided (step S15). When there is any virtual site that needs to be divided (YES), the division unit 15B divides the virtual site into a plurality of subsites and the determination unit 15A determines image pickup conditions for the plurality of subsites (step S16). In addition, at step S16, for any virtual site that does not need to be divided, the determination unit 15A determines, as definitive image pickup conditions for the virtual site, the image pickup conditions provisionally determined at step S15. When there is no virtual site that needs to be divided at step S15 (NO), the image pickup conditions provisionally determined at step S15 are determined as definitive image pickup conditions (step S17).

Note that, at step S15, the determination unit 15A may provisionally determine image pickup conditions by performing image analysis of the endoscope image or may provisionally determine image pickup conditions by comparing the image pickup information and initial image pickup conditions.

Subsequently, the display control unit 16 executes a series of processes for controlling the display device 2 (steps S18, S19, S20, S21, and S22). The display control unit 16 may execute all processes in the series or may execute only some processes in the series. Execution order of the series of processes is not limited to the execution order in an example illustrated in FIG. 5. Processing to be executed by the display control unit 16 may be selected by, for example, the user operating the input instrument 3 (refer to FIG. 1).

In steps S18 and S19, the display control unit 16 causes the display device 2 to display, as image pickup conditions that are preferably satisfied, the image pickup conditions determined by the determination unit 15A. At step S18, when the determination unit 15A determines the image pickup conditions by performing image analysis of the endoscope image at step S15, the display control unit 16 displays at least some of the image pickup conditions. When the determination unit 15A determines the image pickup conditions by comparing the image pickup information and the initial image pickup conditions at step S15, the display control unit 16 displays, as image pickup conditions that are preferably satisfied, at least some of the initial image pickup conditions irrespective of whether the image pickup information satisfies the initial image pickup condition.

Step S19 is executed when the determination unit 15A determines the image pickup conditions by comparing the image pickup information and the initial image pickup conditions at step S15. In this case, the determination unit 15A determines image pickup conditions which satisfy the initial image pickup conditions (steps S16 and S17). At step S19, the display control unit 16 displays the image pickup conditions which satisfy the initial image pickup conditions. Note that, at step S19, the display control unit 16 may display a result of the comparison between the image pickup information and the initial image pickup conditions.

In step S20, when there are a plurality of endoscope images corresponding to one image pickup site, the display control unit 16 causes the display device 2 to display, for each virtual site, the plurality of endoscope images and a plurality of pieces of image pickup information. Note that, when the number of endoscope images corresponding to one image pickup condition is plural, the display control unit 16 may display, as the endoscope images corresponding to the one image pickup condition, endoscope images, the image quality of which is evaluated to be high by the evaluation unit 13A. The display control unit 16 may simultaneously display the plurality of endoscope images or may display the plurality of endoscope images one by one.

In step S21, when an anomaly at an image pickup site is detected by the detection unit 13B, the display control unit 16 causes the display device 2 to display a result of the detection by the detection unit 13B, in other words, existence and details of anomaly.

In step S22, the display control unit 16 causes the display device 2 to display, on the model map based on the time-point information acquired by the acquisition unit 13, an image pickup route when the plurality of endoscope images are picked up.

### Examples of Display Contents

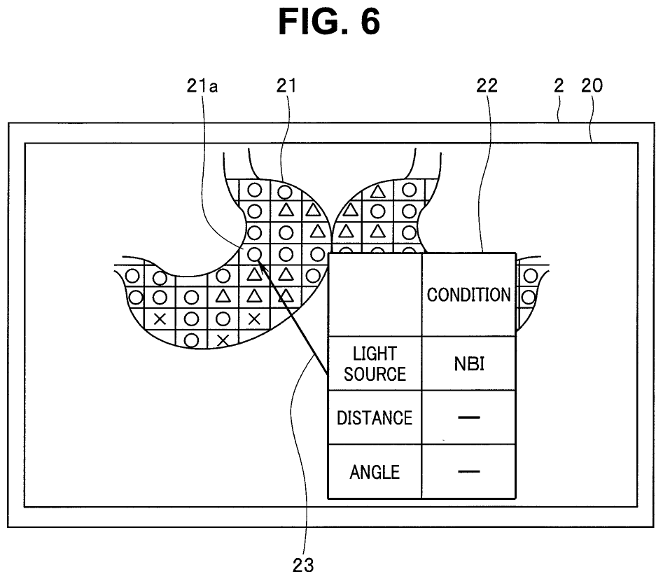

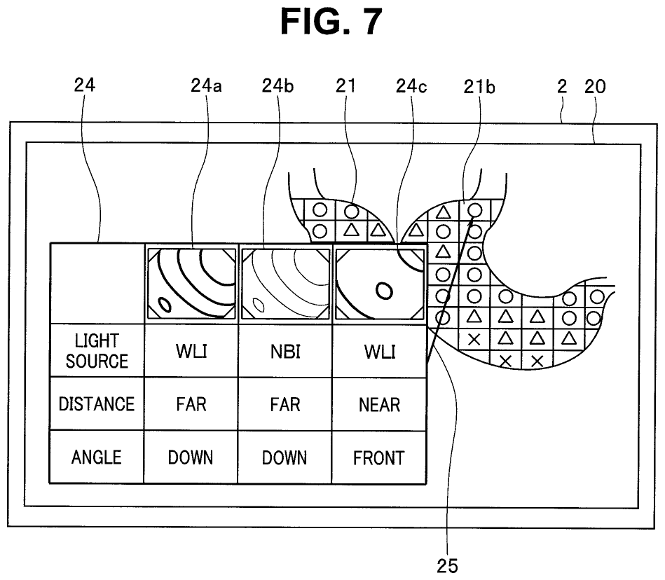

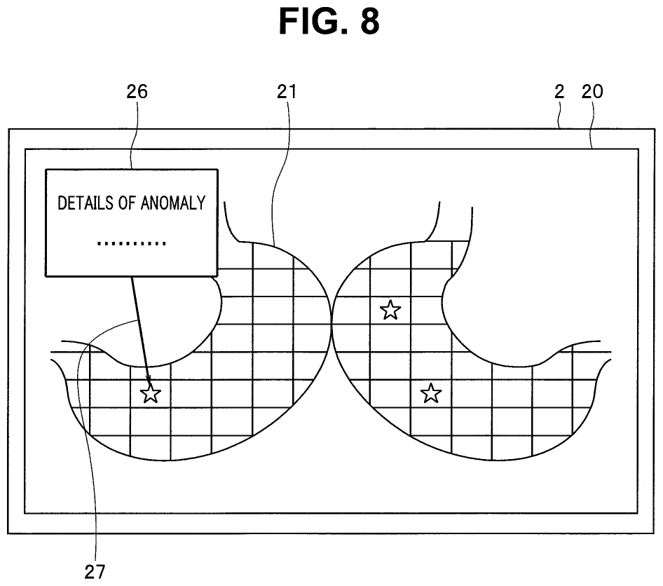

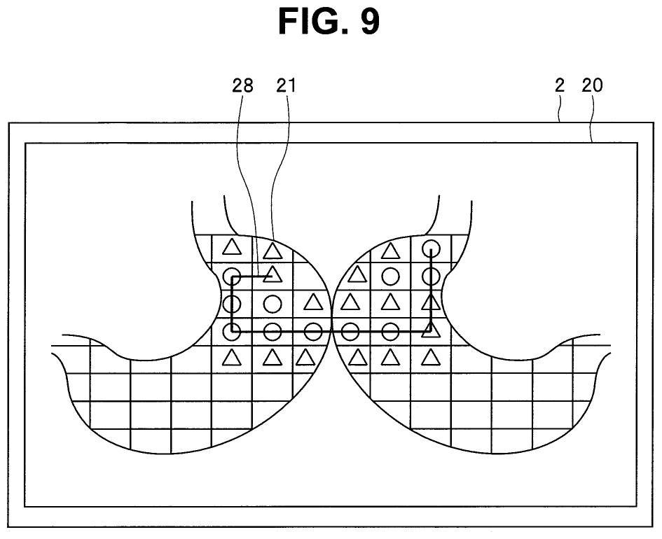

First to fourth examples of contents that the display control unit 16 causes the display device 2 to display (hereinafter referred to as display contents) will be described below. The description will be made on an example in which a subject is stomach. In the first to fourth examples, the display control unit 16 displays a schema diagram of the stomach as a model map of the subject and also displays a plurality of virtual sites. FIG. 6 is an explanatory diagram illustrating the first example of the display contents. FIG. 7 is an explanatory diagram illustrating the second example of the display contents. FIG. 8 is an explanatory diagram illustrating the third example of the display contents. FIG. 9 is an explanatory diagram illustrating the fourth example of the display contents. In FIGS. 6 to 9, reference sign 20 denotes the display unit of the display device 2, and reference sign 21 denotes the model map (schema diagram). In the examples illustrating in FIGS. 6 to 9, each region partitioned in a lattice shape on the model map 21 corresponds to one virtual site.

First, the first example of the display contents will be described below with reference to FIG. 6. The first example is an example in which the display control unit 16 executes the processing at step S19 illustrated in FIG. 5. In the example illustrated in FIG. 6, the display unit 20 displays a table 22 for displaying image pickup conditions. The table 22 includes, as image pickup conditions of a virtual site 21a, an item indicating a condition on the kind of the illumination light (referred to as “light source” in FIG. 6), an item indicating a condition on the distance between the distal end portion 111 and the object (referred to as “distance” in FIG. 6), and an item indicating a condition on the angle between the distal end portion 111 and the object (referred to as “angle” in FIG. 6). In the example illustrated in FIG. 6, only the condition on the kind of the illumination light is displayed among these conditions. In this example, the condition on the kind of the illumination light corresponds to an image pickup condition which satisfies an initial image pickup condition.

An arrow 23 connecting the virtual site 21a and the table 22 indicates that the image pickup conditions displayed in the table 22 are image pickup conditions of the virtual site 21a. A virtual site for which image pickup conditions are displayed may be selected by, for example, the user operating the input instrument 3 (refer to FIG. 1).

In the example illustrated in FIG. 6, a result of the comparison between image pickup information and initial image pickup conditions by the determination unit 15A is displayed for each of a plurality of virtual sites. The comparison result is expressed by, for example, a symbol such as a circle, a triangle, or a cross as illustrated in FIG. 6. The circle expresses, for example, that the image pickup information on the image pickup site corresponding to the virtual site does not satisfy a condition of relatively high importance among the initial image pickup conditions. The triangle expresses, for example, that the image pickup information on the image pickup site corresponding to the virtual site satisfies a condition of relatively high importance among the initial image pickup conditions but does not satisfy a condition of relatively low importance among the initial image pickup conditions. The cross expresses, for example, that the image pickup information on the image pickup site corresponding to the virtual site satisfies all or substantially all initial image pickup conditions. The comparison result may be expressed by a character or a color instead of a symbol.

Note that the display control unit 16 may simultaneously display the model map 21 and the table 22 or may display only one of the model map 21 and the table 22.

The second example of the display contents will be described below with reference to FIG. 7. The second example is an example in which the display control unit 16 executes the processing at step S20 illustrated in FIG. 5. In the example illustrated in FIG. 7, the display unit 20 displays a table 24 for displaying endoscope images and image pickup information. The table 24 displays three endoscope images 24a, 24b, and 24c at the image pickup site corresponding to a virtual site 21b and three pieces of image pickup information corresponding to the three endoscope images 24a, 24b, and 24c.

In the example illustrated in FIG. 7, information on the kind of the illumination light (referred to as “light source” in FIG. 7), distance information (referred to as “distance” in FIG. 7), and angle information (referred to as “angle” in FIG. 7) are displayed as the image pickup information. The kind of the illumination light is different between the endoscope images 24a and 24b. The distance and angle between the distal end portion 111 and the object are different between the endoscope images 24a and 24c.

An arrow 25 connecting the virtual site 21b and the table 24 indicates that the endoscope images 24a to 24c and the image pickup information displayed in the table 24 are endoscope images and image pickup information on the virtual site 21b. A virtual site for which endoscope images and image pickup information are displayed may be selected by, for example, the user operating the input instrument 3 (refer to FIG. 1).

In the second example, similarly to the first example, a result of the comparison between image pickup information and initial image pickup conditions by the determination unit 15A may be displayed for each of a plurality of virtual sites. In the example illustrated in FIG. 7, similarly to the first example illustrated in FIG. 6, the comparison result is expressed by a symbol. The display control unit 16 may simultaneously display the model map 21 and the table 24 or may display only one of the model map 21 and the table 24.

The third example of the display contents will be described below with reference to FIG. 8. The third example is an example in which the display control unit 16 executes the processing at step S21 illustrated in FIG. 5. In the example illustrated in FIG. 8, a result of the detection by the detection unit 13B, in other words, existence of anomaly is displayed at a virtual site. The detection result is expressed by, for example, a symbol such as a star as illustrated in FIG. 8. A virtual site on which a star is displayed indicates that anomaly occurs at the image pickup site corresponding to the virtual site. A virtual site on which no star is displayed indicates that no anomaly occurs at the image pickup site corresponding to the virtual site. Note that existence of anomaly may be expressed by a character, a color, or an endoscope image instead of a symbol.

In the example illustrated in FIG. 8, the display unit 20 displays a frame 26 for displaying details of anomaly detected by the detection unit 13B. Details of anomaly are displayed in the frame 26. An arrow 27 connecting a virtual site 21c and the frame 26 indicates that the anomaly details displayed in the frame 26 are details of anomaly at the image pickup site corresponding to the virtual site 21c. A virtual site for which details of anomaly are displayed may be selected by, for example, the user operating the input instrument 3 (refer to FIG. 1).

The fourth example of the display contents will be described below with reference to FIG. 9. The fourth example is an example in which the display control unit 16 executes the processing at step S22 illustrated in FIG. 5. In the example illustrated in FIG. 9, an image pickup route 28 when a plurality of endoscope images are picked up is displayed on the model map 21.

In the fourth example, similarly to the first example, a result of the comparison between image pickup information and initial image pickup conditions by the determination unit 15A may be displayed for each of a plurality of virtual sites. The comparison result is expressed by, for example, a symbol such as a circle or a triangle as illustrated in FIG. 9. The circle expresses, for example, that the image pickup information on the image pickup site corresponding to the virtual site satisfies all or substantially all initial image pickup conditions. The triangle expresses, for example, that the image pickup information on the image pickup site corresponding to the virtual site satisfies some of the initial image pickup conditions. In the fourth example, start and end points of the image pickup route 28 may be expressed by, for example, symbols.

### Operations and Effects

Operations and effects of the image processing apparatus 1, the image processing method, and the image processing program according to the present embodiment will be described below. In the present embodiment, the acquisition unit 13 acquires image pickup information from examination information, and the production unit 15 associates the image pickup information with a virtual site. According to the present embodiment, the image pickup information associated with a virtual site can be used to determine whether to perform image pickup again at the image pickup site corresponding to the image pickup information, and as a result, it is possible to prevent image pickup omission and image pickup failure at a site where image pickup needs to be performed.

In the present embodiment, the division unit 15B divides, for example, the virtual site corresponding to an image pickup site at which anomaly such as a lesion is detected into a plurality of subsites (refer to steps S15 and S16 in FIG. 5). According to the present embodiment, an image pickup site at which anomaly is detected can be examined intensively by, for example, dividing the site into a subsite including anomaly and a subsite not including anomaly and additionally determining an image pickup condition for the subsite including anomaly.

In the present embodiment, it is possible to display image pickup conditions which satisfy initial image pickup conditions (refer to step S19 in FIG. 5 and FIG. 6). Thus, according to the present embodiment, it is possible to prevent image pickup failure.

In the present embodiment, a plurality of endoscope images and a plurality of pieces of image pickup information can be displayed for each virtual site (refer to step S20 in FIG. 5 and FIG. 7). According to the present embodiment, it is possible to perform accurate diagnosis by referring to the plurality of endoscope images and the plurality of pieces of image pickup information.

Note that, in the example illustrated in FIG. 7, information on the kind of the illumination light, distance information, and angle information are displayed as the plurality of pieces of image pickup information, and the endoscope images 24a and 24b between which the kind of the illumination light is different and the endoscope images 24a and 24c between which the distance and angle between the distal end portion 111 and the object are different are displayed as the plurality of endoscope images. In the present embodiment, image pickup information other than the above-described information can be displayed as the plurality of pieces of image pickup information, and a plurality of endoscope images among which the image pickup information other than the above-described information is different can be displayed as the plurality of endoscope images. Specifically, the display control unit 16 can display, as image pickup information, for example, information on the optical magnification, information on the electronic magnification, information on the light quantity of the illumination light, information on whether a region is sprayed with pigment, and information on existence and kind of a treatment instrument.

In the present embodiment, existence and details of anomaly can be displayed (refer to step S21 in FIG. 5 and FIG. 8). Thus, according to the present embodiment, an image pickup site at which anomaly is detected can be examined intensively.

In the present embodiment, an image pickup route when a plurality of endoscope images are picked up can be displayed (refer to step S22 in FIG. 5 and FIG. 9). Thus, according to the present embodiment, it is possible to provide feedback on an examination procedure, thereby improving skills of the user.

The present invention is not limited to the above-described embodiment but may be provided with various changes, modifications, and the like without departing from the gist of the present invention. For example, the image processing apparatus, the image processing method, and the image processing program of the present invention are also applicable not only to medical fields but also to industrial fields.

What is claimed is:

1. An image processing apparatus comprising a processor, wherein the processor is configured to:
acquire an endoscope image obtained through image pickup of a subject by an endoscope;
estimate an image pickup site in the subject of the endoscope image;
associate the image pickup site of the endoscope image with a site corresponding to the image pickup site on an organ model map based on a result of the estimation, the model map including an image pickup condition determined for each site on the map;
output the model map and the endoscope image to a monitor; and
output, to the monitor, the image pickup condition associated with the site on the model map, the site corresponding to the image pickup site of the endoscope image outputted on the monitor.

2. The image processing apparatus according to claim 1, wherein the processor is configured to estimate the image pickup site by performing image analysis of the endoscope image.

3. The image processing apparatus according to claim 1, wherein the processor is configured to acquire image pickup information corresponding to the endoscope image and indicating a state of at least one of the endoscope or the subject when the endoscope image is picked up.

4. The image processing apparatus according to claim 3, wherein the processor is configured to:
further acquire system information related to operation of the endoscope, and
acquire the image pickup information from the system information.

5. The image processing apparatus according to claim 3, wherein the processor is configured to acquire the image pickup information by performing image analysis of the endoscope image.

6. The image processing apparatus according to claim 1, wherein the processor is configured to store an initial image pickup condition defined in advance, the initial image pickup condition being a condition for image pickup of the subject.

7. The image processing apparatus according to claim 1, wherein the processor is further configured to:
divide a site on the model map into a plurality of subsites,
determine the image pickup condition for each of the plurality of subsites, and
output at least one of the image pickup conditions to the monitor.

8. The image processing apparatus according to claim 3, wherein
the processor is configured to use a first memory configured to store the endoscope image and a second memory configured to store the image pickup information, and
the processor is configured to read, from the first memory, the endoscope image in plurality that corresponds to the image pickup site and at least part of the image pickup information corresponding to which is mutually different, read the image pickup information in plurality corresponding to the endoscope image in plurality from the second memory, and output at least one of the endoscope image in plurality or the image pickup information in plurality to the monitor.

9. The image processing apparatus according to claim 1, wherein
the processor is configured to use a first memory configured to store the endoscope image,
the processor is configured to evaluate image quality of the endoscope image by performing image analysis of the endoscope image and store a result of the evaluation and the endoscope image in association in the first memory, and
the processor is configured to read, from the first memory, the endoscope image having a predetermined image quality from among the endoscope image in plurality corresponding to the image pickup site and output the endoscope image read to the monitor.

10. The image processing apparatus according to claim 1, wherein the processor is configured to detect anomaly at the image pickup site by performing image analysis of the endoscope image and output a result of the detection to the monitor.

11. The image processing apparatus according to claim 1, wherein
the processor is configured to further acquire time-point information on a time point at which the endoscope image is picked up, and
the processor is configured to output, to the model map on the monitor based on the time-point information, an image pickup route when the endoscope image in plurality is picked up.

12. An image processing method, wherein a processor is configured to:
acquire an endoscope image obtained through image pickup of a subject by an endoscope;
estimate an image pickup site in the subject of the endoscope image; and
associate, based on a result of the estimation, the image pickup site with an organ model map including an image pickup condition defined in advance which is associated with a particular site, output the model map and the endoscope image to a monitor, and output the image pickup condition in the model map to the monitor, the image pickup condition being associated with an image pickup position of the endoscope image outputted on the monitor.

13. A non-transitory storage medium storing a computer program configured to cause a computer to:
acquire an endoscope image obtained through image pickup of a subject by an endoscope;
estimate an image pickup site in the subject of the endoscope image;
associate, based on a result of the estimation, the image pickup site with an organ model map including an image pickup condition defined in advance which is associated with a particular site; and
output the model map and the endoscope image to a monitor and output the image pickup condition in the model map to the monitor, the image pickup condition being associated with an image pickup position of the endoscope image outputted on the monitor.
